(19) United States
(12) Patent Application Publication
(10) Pub. No.: US 2012/0135965 A1
(43) Pub. Date: May 31, 2012

(54) AMORPHOUS FOSAMPRENAVIR CALCIUM

(75) Inventors: Satish Manohar Bhoge, Ahmed Nagar, Maharashtra (IN); Prakash Kshirsagar, Pune (IN); Santosh Richhariya, Sagar (IN); Anshul Agrawal, Ghaziabad (IN); Kaptan Singh, Ghaziabad (IN)

(73) Assignee: RANBAXY LABORATORIES LIMITED, New Delhi, Delhi (IN)

(21) Appl. No.: 13/320,991

(22) PCT Filed: May 20, 2010
(86) PCT No.: PCT/IB2010/052251

§ 371 Date: Jan. 27, 2012

(30) Foreign Application Priority Data
May 20, 2009 (IN) 1035/DEL/2009

(51) Int. Cl. A61K 31/665; A61P 31/18; C07F 9/655

(52) U.S. Cl. 514/99; 549/222

(57) ABSTRACT

The present invention relates to amorphous Fosamprenavir calcium and processes for its preparation, a pharmaceutical composition comprising it and a method for treating a HIV infection therewith.

### FIELD OF THE INVENTION

The present invention relates to amorphous Fosamprenavir calcium and processes for its preparation.

### BACKGROUND OF THE INVENTION

Fosamprenavir calcium is chemically (3S)-tetrahydrofuran-3-yl (1S ,2R)-3-[[(4-aminophenyl) sulfonyl](isobutyl)amino]-1-benzyl-2-(phosphonooxy)propylcarbamate monocalcium salt of Formula I.

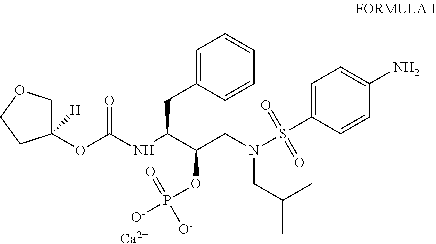

Fosamprenavir calcium is a prodrug of amprenavir, an inhibitor of HIV protease. It is useful in combination with other antiretroviral agents for the treatment of human immunodeficiency virus (HIV-1) infection.

Preparation of fosamprenavir or its salts in solid forms has been mentioned as a critical problem in the prior art. Antimicrob. Agents Chemother., 2004, 48(3), 791-798 says that though the free acid and sodium salt of fosamprenavir are the most soluble over the relevant physiological pH range, neither could be isolated as a crystalline solid. Antimicrob. Agents Chemother., 2004, 48(3), 791-798 also says that the sodium salt is extremely hygroscopic. According to above reference, the calcium salt has reduced solubility compared to the other forms, but it could be produced as a crystalline form. J. Clin. Pharmacol. 2002; 42; 887-898 says that, as the calcium salt could be crystallized, it was the only acceptable choice for a formulation.

U.S. Pat. No. 6,514,953 provides processes for the preparation of crystalline form I of fosamprenavir calcium. U.S. Pat. No. 6,514,953 says that a range of salts of fosamprenavir were made including di-sodium, di-potassium, magnesium, zinc, ethylene diamine, piperazine and of these, the piperazine salt was a crystalline solid, but had the practical disadvantage of likely toxicity at the anticipated dose. U.S. Pat. No. 6,514,953 further says that the calcium salt, calcium (3S) tetrahydro-3-furanyl (1S,2R)-3-[[(4-aminophenyl)sulfonyl](isobutyl)amino]-1-benzyl-2-phosphonooxy)propylcarbamate, was surprisingly found to have a stable crystalline form.

### SUMMARY OF THE INVENTION

The present inventors have found that fosamprenavir calcium can be prepared in amorphous form. The present inventors have also observed that the amorphous fosamprenavir calcium has appreciable solubility over the relevant physiological pH range. The solubility of amorphous fosamprenavir calcium is superior to that of crystalline form I of fosamprenavir calcium. Further, the amorphous fosamprenavir calcium of the present invention is essentially non-hygroscopic, stable on storage, reproducible and suitable for developing pharmaceutical dosage forms.

The term “charging” according to the present inventions includes loading, feeding, adding, filling and/or infusing.

The term “collecting” according to the present inventions includes unloading, amassing, gathering, scaling and/or piling.

### BRIEF DESCRIPTION OF THE DRAWINGS

FIG. 1 depicts the XRPD (X-Ray Powder Diffractogram) of amorphous fosamprenavir calcium obtained according to Example 1.

FIG. 2 depicts the XRPD (X-Ray Powder Diffractogram) of amorphous fosamprenavir calcium obtained according to Example 2.

FIG. 3 depicts the XRPD (X-Ray Powder Diffractogram) of amorphous fosamprenavir calcium obtained according to Example 4.

FIG. 4 depicts the XRPD (X-Ray Powder Diffractogram) of crystalline form I of fosamprenavir calcium obtained according to Example 5.

FIG. 4A provides the table of the XRPD (X-Ray Powder Diffractogram) of crystalline form I of fosamprenavir calcium obtained according to Example 5.

### DETAILED DESCRIPTION OF THE INVENTION

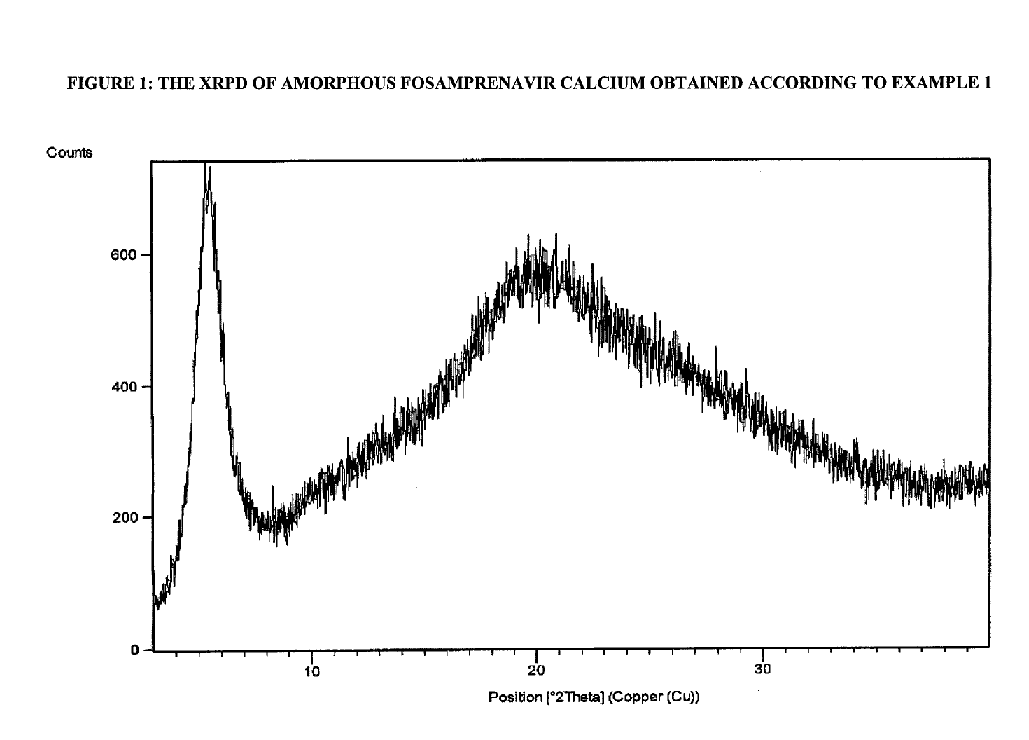

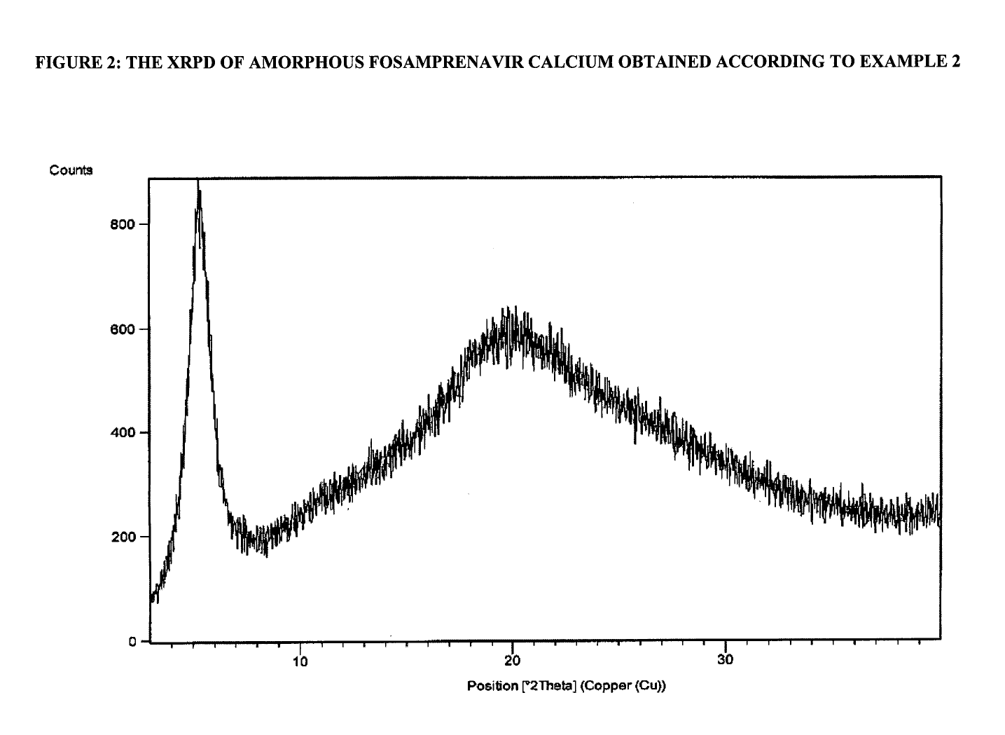

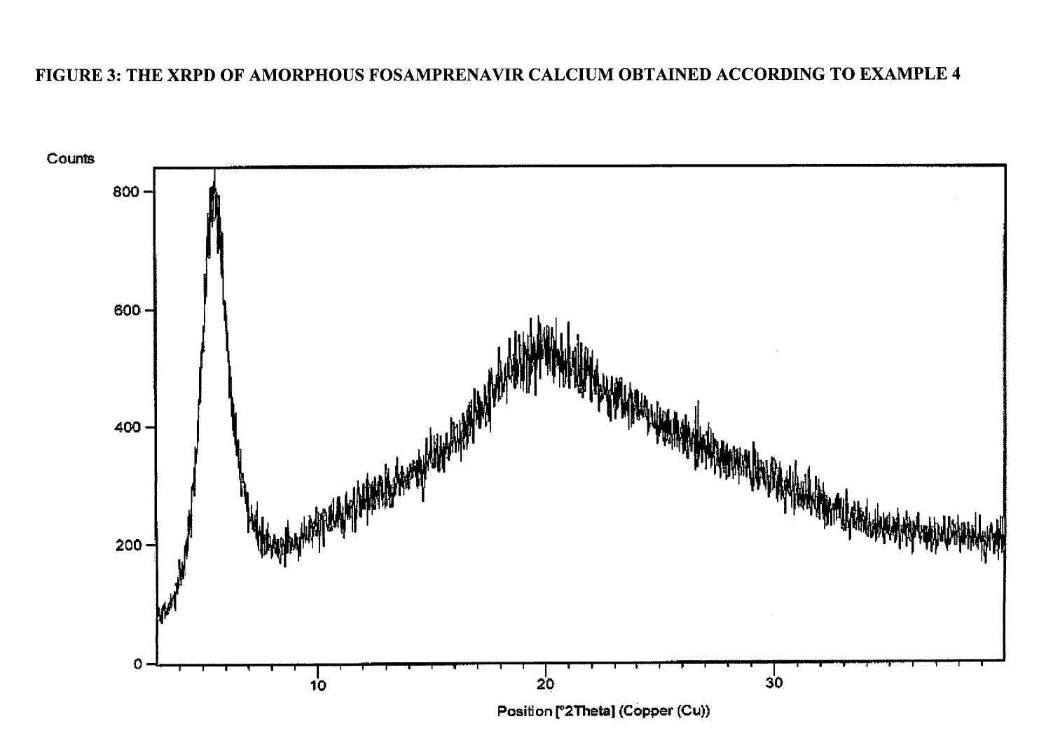

A first aspect of the present invention provides amorphous fosamprenavir calcium. The amorphous fosamprenavir calcium has substantially the same XRPD pattern as depicted in FIG. 1, FIG. 2 or FIG. 3 of the accompanied drawing. The amorphous fosamprenavir calcium of the present invention is substantially soluble over the pH range of about 3 to about 5.5. The amorphous fosamprenavir calcium requires not more than about 200 ml of aqueous solution having a pH of about 3 to about 5.5 for dissolving about 1 g of amorphous fosamprenavir calcium. The amorphous fosamprenavir calcium of the present invention is essentially non-hygroscopic. The amorphous fosamprenavir calcium has an increase in mass of not more than about 19% when stored at 25±1° C. at 80±2% RH (Relative Humidity) for about 24 hours. For example, the amorphous fosamprenavir calcium has an increase in mass of about 16% to about 18% when stored at 25±1° C. at 80±2% RH (Relative Humidity) for about 24 hours. The amorphous fosamprenavir calcium of the present invention is stable. The amorphous fosamprenavir calcium is not converted into any crystalline form on storage, for example, on storage at about 40±1° C. at 75±2% RH (Relative Humidity) for about 1 month or above, for example, about two months.

A second aspect of the present invention provides a process for the preparation of amorphous fosamprenavir calcium, wherein the process comprises,
a) charging a solution of fosamprenavir calcium to a thin film dryer,
b) removing the solvent from the solution of fosamprenavir calcium by thin film drying, and
c) collecting amorphous fosamprenavir calcium from the thin film dryer.

The starting fosamprenavir calcium may be prepared according to the methods provided in, for example, U.S. Pat. No. 6,514,953. The solution of fosamprenavir calcium may be obtained directly from a reaction mixture in which fosamprenavir calcium is formed or it may be prepared by dissolving fosamprenavir calcium in a solvent. The solvent may be, for example, methanol, N,N-dimethylformamide, dimethylsulphoxide, ethanol, isopropanol, tetrahydrofuran, acetone, ethyl acetate, dichloromethane or a mixture thereof. The solution may be optionally filtered to remove any undissolved material. The solution of fosamprenavir calcium is charged to a thin film dryer, for example, BUCHI Rotavapor®. The solvent is removed from the solution by thin film drying. The drying process may be accompanied by heating at a temperature of about 35° C. or above, for example, about 80° to about 85° C. The feeding rate of the solution is controlled in such a way to facilitate the thin film formation and the evaporation rate. The vapor duct of the thin film dryer may optionally have a sealing system so that the drying is carried under vacuum. The amorphous fosamprenavir calcium is collected from the thin film dryer. The amorphous fosamprenavir calcium may optionally be further dried under vacuum to reduce residual solvent content.

A third aspect of the present invention provides a process for the preparation of amorphous fosamprenavir calcium, wherein the process comprises,
a) charging a solution of fosamprenavir calcium to a spray dryer,
b) removing the solvent from the solution of fosamprenavir calcium by spray drying, and
c) collecting amorphous fosamprenavir calcium from the spray dryer.

The starting fosamprenavir calcium may be prepared according to the methods provided in the prior art, for example, U.S. Pat. No. 6,514,953. The solution of fosamprenavir calcium may be obtained directly from a reaction mixture in which fosamprenavir calcium is formed or it may be prepared by dissolving fosamprenavir calcium in a solvent. The solvent may be, for example, methanol, N,N-dimethylformamide, dimethylsulphoxide, ethanol, isopropanol, tetrahydrofuran, acetone, ethyl acetate, dichloromethane or a mixture thereof. The solution may be optionally filtered to remove any undissolved material. The solution of fosamprenavir calcium is charged to a spray dryer. The inlet and outlet temperatures, feed rate, and atomizer type can be adjusted to optimize output and particle size. The air inlet temperature may be controlled from about 70° to about 90° C. The outlet temperature may be controlled from about 35° to about 55° C. An inert gas, for example, nitrogen gas may be used as a carrier gas. After the drying process, the amorphous fosamprenavir calcium is collected from the spray dryer and optionally further dried under vacuum to reduce residual solvent content.

A fourth aspect of the present invention provides a process for the preparation of amorphous fosamprenavir calcium, wherein the process comprises,

a) forming a solution of fosamprenavir calcium in a solvent,

b) treating the solution of step a) with an antisolvent, and

c) isolating amorphous fosamprenavir calcium from the mixture thereof.

The starting fosamprenavir calcium may be prepared according to the methods provided in the prior art, for example, U.S. Pat. No. 6,514,953. The solution of fosamprenavir calcium may be formed in the reaction mixture of preparing fosamprenavir calcium or it may be prepared by dissolving fosamprenavir calcium in a solvent. The solvent may be, for example, methanol, N,N-dimethylformamide, dimethylsulphoxide, ethanol, isopropanol, tetrahydrofuran, acetone, ethyl acetate, dichloromethane or a mixture thereof. The solution may be optionally filtered to remove any undissolved material. The solution of fosamprenavir calcium is treated with an antisolvent. The antisolvent may be a hydrocarbon, for example, n-pentane, n-hexane, n-pentane, heptane, hexanes, cyclohexane or a mixture thereof. The treatment with the antisolvent may be carried out, for example, by adding the solution of fosamprenavir calcium into the antisolvent. The treatment with the antisolvent may be completed, for example, in about 5 minutes to about 15 minutes. The treatment with the antisolvent may be followed by stirring the mixture for about 1 minute to about 100 hours, for example, about 1 hour to about 5 hours. The stirring may be carried out at about 0° to about 50° C., for example, at about 15° to about 30° C. The amorphous fosamprenavir calcium so obtained may be isolated from the mixture by the methods including concentration, distillation, decantation, filtration, evaporation, centrifugation or a combination thereof.

A fifth aspect of the present invention provides a pharmaceutical composition comprising amorphous fosamprenavir calcium and a pharmaceutically acceptable carrier.

A sixth aspect of the present invention provides a method treating a HIV infection, which comprises administering a therapeutically effective amount of amorphous fosamprenavir calcium to a patient in need thereof.

The XRPD of the samples were determined by using Panalytical X'Pert Pro X-Ray Powder Diffractometer in the range 3 to 40 degree 2 theta with a step size of 0.02 and under tube voltage and current of 45 Kv and 40 mA respectively. Copper radiation of wavelength 1.54 angstrom and Xceletor detector were used.

While the present invention has been described in terms of its specific embodiments, certain modifications and equivalents will be apparent to those skilled in the art and are intended to be included within the scope of the present invention.

### EXAMPLES

### Example 1

### Preparation of Amorphous Fosamprenavir Calcium

Fosamprenavir calcium (5 g) was added to methanol (125 ml), stirred at 25° to 30° C. and filtered to remove any undissolved material. The filtered solution was fed to a BUCHI Rotavapor® (Model No. R-205; 500 ml) in small lots so as to form a thin film. The solvent was evaporated at 80° to 85° C. under vacuum (1 to 2 mmHg). The solid residue was further stirred for 30 minutes at 80° to 85° C. under vacuum (1 to 2 mmHg). The solid so obtained was collected from the BUCHI Rotavapor® and dried at 55° to 60° C. under vacuum (10 to 15 mm Hg) for 10 to 12 hours to obtain the title compound having an XRPD pattern as depicted in FIG. 1.

### Yield: 2.7 g

### Example 2

### Preparation of Amorphous Fosamprenavir Calcium

Fosamprenavir calcium (5 g) was dissolved in methanol (125 ml) at 25° to 30° C. The solution was filtered and fed to a spray dryer (BUCHI, B-290) at feed pump RPM of 1% to 3%. The following parameters were controlled in the spray drying process:
Nozzle Diameter: 0.7 mm
Carrier gas: Nitrogen at 2.0 to 3.0 kg/cm²
Air inlet temperature: 80° to 85° C.
Outlet temperature: 35° to 55° C.
Type of atomizer: Two fluid nozzle
The solvent was evaporated at 80° to 85° C. by spray drying. The solid so obtained was collected from the spray dryer and further dried at 55° to 60° C. under vacuum (10 to 15 mmHg) for 10 to 12 hours to obtain the title compound having an XRPD pattern as depicted in FIG. 2. The compound so obtained was stored at 40±1° C. at 75±2% RH for two months and no change in the XRPD pattern was observed.

### Yield: 2.5 g

### Example 3

### Preparation of Amorphous Fosamprenavir Calcium

Fosamprenavir calcium (100 g) was dissolved in methanol (1300 ml) at 25° to 30° C. The solution was filtered, washed with methanol (200 ml) at 25° to 30° C. and fed to a spray dryer (BUCHI, B-290) at feed pump RPM of 1% to 3%. The following parameters were controlled in the spray drying process:
Nozzle Diameter: 0.7 mm
Carrier gas: Nitrogen at 2.0 to 3.0 kg/cm²
Air inlet temperature: 75° to 80° C.
Outlet temperature: 35° to 50° C.
Type of atomizer: Two fluid nozzle
The solvent was evaporated at 75° to 80° C. by spray drying. The solid so obtained was collected from the spray dryer and further dried at 35° to 40° C. under vacuum (10 to 15 mmHg) to obtain the title compound.

### Yield: 85 g

### Example 4

### Preparation of Amorphous Fosamprenavir Calcium

Fosamprenavir calcium (5 g) was added to methanol (25 ml) at 25° to 30° C. The temperature was raised to 40° to 45° C. to obtain a solution. The solution was added into n-pentane (100 ml) in 10 minutes at 25° to 30° C. and stirred for 1 hour to 2 hours at 25° to 30° C. The solid was filtered, washed with n-pentane (10 ml) at 25° to 30° C. and dried at 35° C. for 15 hours to obtain the title compound having an XRPD pattern as depicted in FIG. 3.

### Yield: 3.0 g

### Example 5

### Preparation of Crystalline Form I of Fosamprenavir Calcium

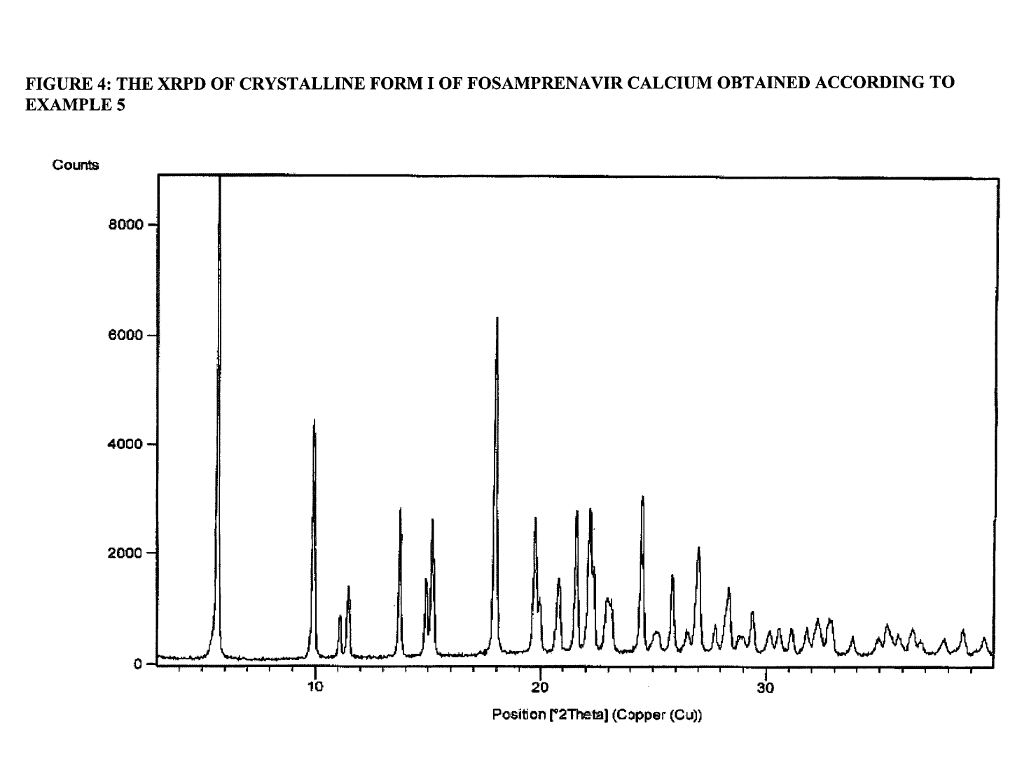

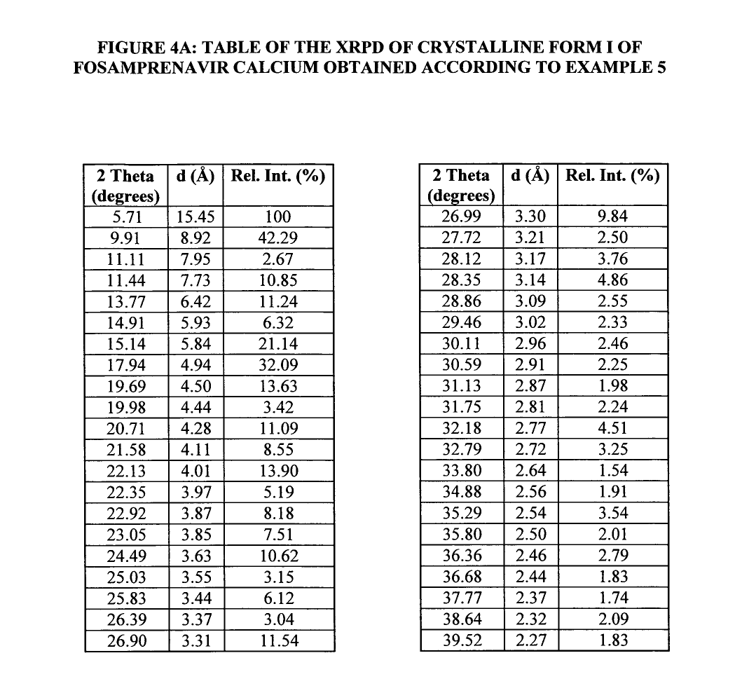

Fosamprenavir calcium (100 g) was mixed with ethanol (1800 ml) at 25° to 30° C. and the temperature was raised to 70° to 75° C. to obtain a solution. The solution was stirred for 30 minutes at 70° to 75° C. Activated charcoal (5 g) was added to the solution at 70° to 75° C. and stirred further for 30 minutes at 70° to 75° C. The mixture was subjected to hot filtration and washed with ethanol (200 ml). The temperature of filtrate was raised to 70° to 75° C. and stirred for 10 minutes at 70° to 75° C. De-ionized water (300 ml) was added slowly at 70° to 75° C. and the mixture was cooled slowly to 25° to 30° C. followed by stirring for 3 hours to 4 hours at 25° to 30° C. The solid obtained was filtered and washed with a mixture of ethanol (100 ml) and deionized water (100 ml) at 25° to 30° C. The solid was further washed with de-ionized water (200 ml) and dried under vacuum at 35° to 40° C. to obtain the title compound having an XRPD pattern as depicted in FIG. 4.

### Yield: 90 g

TABLE 1
COMPARISON OF AQUEOUS SOLUBILITY OF AMORPHOUS
FOSAMPRENAVIR CALCIUM AND CRYSTALLINE
FORM I OF FOSAMPRENAVIR CALCIUM

| | | Amorphous fosamprenavir | | Crystalline form I of | |
|---|---|---|---|---|---|
| | | calcium | | fosamprenavir calcium | |
| | | Parts of solvent | Solubility* | Parts of solvent | Solubility* |
| | | required per g of | (mg/ml of | required per g of | (mg/ml of |
| S. No. | pH | solute | solvent) | solute | solvent) |
| 1. | 1.47 | 800 | 1.25 | 2100 | 0.5 |
| 2. | 3.27 | 100 | 10 | 400 | 2.5 |
| 3. | 4.11 | 150 | 6.7 | 250 | 4.0 |
| 4. | 5.02 | 150 | 6.7 | 300 | 3.3 |
| 5. | 6.27 | Less than 10,000 | 0.1 | 10,000** | — |
| | | but more than 5,000 | | | |

*Solubility test was performed according to Unites States Pharmacopeia 32 and mg/ml of solvent was by qualitative observation.
**Not soluble up to 10,000 parts.

TABLE 2
COMPARISON OF HYGROSCOPICITY OF AMORPHOUS
FOSAMPRENAVIR CALCIUM AND CRYSTALLINE
FORM I OF FOSAMPRENAVIR CALCIUM

| | | Hygroscopicity* |
|---|---|---|
| | Fosamprenavir | % increase in mass at |
| S. No. | calcium | 25 ± 1° C./80 ± 2% RH for 24 h |
| 1. | Amorphous | 17.5 |
| 2. | Crystalline form I | 11.62 |

*Hygroscopicity was determined according to European Pharmacopoeia 6.0.

1. Amorphous fosamprenavir calcium.

2. Amorphous fosamprenavir calcium according to claim 1 having substantially the same XRPD pattern as depicted in FIG. 1, FIG. 2 or FIG. 3 of the accompanied drawing.

3. A process for the preparation of amorphous fosamprenavir calcium, wherein the process comprises,
a) forming a solution of fosamprenavir calcium in a solvent;
b) isolating fosamprenavir calcium from the solution by using a suitable separation technique; and
c) collecting amorphous fosamprenavir calcium.

4. A process according to claim 3, wherein the process comprises,
a) charging a solution of fosamprenavir calcium to a dryer selected from spray dryer or thin film dryer;
b) removing the solvent from the solution of fosamprenavir calcium by spray drying or thin film drying; and
c) collecting amorphous fosamprenavir calcium from the dryer.

5. A process according to claim 3, wherein the process comprises,
a) treating the solution of step a) with an antisolvent, and
b) isolating amorphous fosamprenavir calcium from the mixture.

6. A process according to claim 3, 4 or 5, wherein the solvent is methanol, N,N-dimethylformamide, dimethylsulphoxide, ethanol, isopropanol, tetrahydrofuran, acetone, ethyl acetate, dichloromethane or a mixture thereof.

7. A process according to claim 5, wherein the antisolvent is a hydrocarbon.

8. A process according to claim 7, wherein the hydrocarbon is n-pentane, n-hexane, n-pentane, heptane, hexanes, cyclohexane or a mixture thereof.

9. A pharmaceutical composition comprising amorphous fosamprenavir calcium and a pharmaceutically acceptable carrier.

10. A method treating a HIV infection, which comprises administering a therapeutically effective amount of amorphous fosamprenavir calcium to a patient in need thereof.
